(19) United States
(12) Patent Application Publication
(10) Pub. No.: US 2017/0333711 A1
(43) Pub. Date: Nov. 23, 2017

(54) DEVICE FOR EFFECTIVE NON-INVASIVE DESYNCHRONIZING NEUROSTIMULATION

(71) Applicant: Forschungszentrum Juelich GmbH, Juelich (DE)

(72) Inventors: Peter Alexander Tass, Juelich (DE); Oleksandr POPOVYCH, Dueren (DE); Markos XENAKIS, Athens (GR)

(21) Appl. No.: 15/522,334

(22) PCT Filed: Oct. 30, 2015
(86) PCT No.: PCT/EP2015/075297

§ 371 Date: Apr. 27, 2017

(30) Foreign Application Priority Data
Nov. 3, 2014 (DE) 10 2014 115 997.7

(51) Int. Cl. A61N 1/36; A61B 5/00; A61B 5/0476

(52) U.S. Cl. CPC A61N 1/36025; A61B 5/4082; A61B 5/4064; A61B 5/0476; A61N 1/3603

(57) ABSTRACT

A device that suppresses a pathological synchronous and oscillatory neuron activity, and includes a non-invasive stimulation for stimulation, using stimuli, of neurons in the patient's brain and/or spinal cord, where those neurons are showing pathologically synchronous and oscillatory neuron activity, and the stimuli are designed to suppress are this activity when administered to the patient. Moreover, a measuring unit records measurement signals reflecting the neuron activity of the stimulated neurons and a control and analysis unit controls the stimulation unit to administer stimuli, check the success of stimulation based on the measurement, and, if the stimulation success is not sufficient, insert one or more stimulation breaks in the application of the stimuli or extend one or more stimulation breaks, where no stimuli that could suppress the pathological synchronous and oscillatory neuron activity are applied during the stimulation breaks.

### CROSS-REFERENCE TO RELATED APPLICATIONS

This application is a National Stage entry of International Application No. PCT/EP2015/075297, filed Oct. 30, 2015, which claims priority to German Patent Application No. 10 2014 115 997.7 filed Nov. 3, 2014, the disclosures of these priority applications are incorporated in their entirety herein by reference.

### TECHNICAL FIELD

The invention relates to an apparatus and to a method for effective non-invasive desynchronizing neurostimulation.

### BACKGROUND

Nerve cell assemblies in the circumscribed regions of the brain are pathologically, e.g. excessively, synchronously, active in patients with neurological or psychiatric diseases such as Parkinson's disease, essential tremor, dystonia, functional disturbances after a stroke, migraine, obsessive compulsive disorders, epilepsy, tinnitus, schizophrenia, borderline personality disturbance and irritable bowel syndrome. In this case, a large number of neurons synchronously form action potentials, i.e. the participating neurons fire excessively synchronously. In a healthy person, in contrast, the neurons fire with a different quality, i.e. in an uncorrelated manner, in these brain sectors.

In Parkinson's disease, the pathologically synchronous activity changes the neuronal activity in other brain regions, e.g. in areas of the cerebral cortex such as the primary motor cortex. In this respect, the pathologically synchronous activity in the region of the thalamus and of the basal ganglia, for example, imposes its rhythm on the cerebral cortex areas such that ultimately the muscles controlled by these areas develop pathological activity, e.g. a rhythmic trembling (tremor).

With chronically subjective tinnitus, pathological synchronous activity is found in a network of auditory and non-auditory brain areas.

In patients with brain diseases and/or spinal cord diseases that are characterized by excessively synchronized neuronal activity, non-invasively determined spatiotemporal stimulus patterns, in particular “coordinated reset” stimulation (CR stimulation) are applied to achieve permanent relief. The non-invasive CR stimulation can be implemented by means of different stimulation modes;

(i) by sensory stimulation, i.e. by physiological stimulation of receptors such as acoustic stimulation of the inner ear, visual stimulation of the retina or mechanical (e.g. vibrotactile) or thermal stimulation of receptors of the skin, hypoderm, muscles and sinews;

(ii) by stimulation of peripheral nerves (and associated receptors) e.g. by means of electric current (e.g. transcutaneous electrostimulation), by means of magnetic fields (transdermal magnetic stimulation) or by means of ultrasound; and

(iii) by stimulation of the brain or spinal cord e.g. by means of electric current (e.g. external cranial or transcranial neurostimulation), by means of magnetic fields (e.g. transcranial magnetic stimulation) or by means of ultrasound.

Acoustic CR stimulation is used to treat chronically subjective tonal or narrow-band tinnitus. For this purpose, therapeutic sounds are adapted to the dominant tinnitus tone and are applied in the sense of CR stimulation to achieve a long-lasting desynchronization of the pathologically synchronous activity or even a lasting desynchronization thereof that considerably survives the switching off of the stimulation. The acoustic CR stimulation for treating tinnitus effects a significant and considerably pronounced reduction of the symptoms (cf. P. A. Tass, I. Adamchic, H.-J. Freund, T. von Stackelberg, C. Hauptmann: Counteracting tinnitus by acoustic coordinated reset neuromodulation. Restorative Neurology and Neuroscience 30, 137-159 (2012)), a significant reduction in pathological neuronal synchronization in a network of auditory and non-auditory brain areas (cf. P A. Tass, I. Adamchic, H.-J. Freund, T. von Stackelberg, C. Hauptmann: Counteracting tinnitus by acoustic coordinated reset neuromodulation. Restorative Neurology and Neuroscience 30, 137-159 (2012). I. Adamchic, T. Toth, C. Hauptmann, P A. Tass: Reversing pathologically increased EEG power by acoustic CR neuromodulation. Human Brain Mapping 35 (2014) 2099-2118), a significant reduction of the pathological interactions between different brain areas therein (cf. A. N. Silchenko, I. Adamchic, C. Hauptmann, P. A. Tass: Impact of acoustic coordinated reset neuromodulation on effective connectivity in a neural network of phantom sound. Neuroimage 77, 133-147 (2013)). as well as in different frequency ranges (cf. I. Adamchic, B. Langguth, C. Hauptmann, P. A. Tass: Abnormal brain activity and cross-frequency coupling in the tinnitus network. Frontiers in Neuroscience 8, 284 (2014)).

Parkinson's disease can be treated in an analog manner by means of vibrotactile CR stimulation. Further indications are e.g. represented by epileptic fits, functional disturbances after stroke, chronic pain syndromes (by means of vibrotactile and/or thermal CR stimulation), migraine (e.g. by means of visual CR stimulation). These diseases can furthermore be treated by transcranial magnetic stimulation or by direct electrical stimulation of the brain or direct brain stimulation by means of ultrasound.

Stimulation should be able to take place using stimuli of smaller intensity for the above-listed stimulation modalities (i) to (iii) for the reasons listed below to avoid side effects and/or to increase the therapeutic effect;

(i) It is important in sensory stimulation to be able to achieve the desired stimulation effects (e.g. a phase reset of the pathologically synchronized oscillatory activity in the brain or spinal cord) at all with as small a stimulus level as possible. E.g. hard-of-hearing patients typically have to be treated in acoustic CR stimulation to treat tinnitus. Stimulation with loud sounds can damage the inner ear, make communication with others more difficult, and mask warning sounds (e.g. vehicle horn, bicycle bell) or can be perceived as very unpleasant by the patient as a result of the intolerability threshold extending comparatively closely to the auditory threshold and the loud stimulation can also be heard by the environment of the patient and can be perceived as disturbing. Unpleasant dazzling effects can in particular occur in the visual CR stimulation of migraine patients. In the mechanical, e.g. vibrotactile or thermal CR stimulation of patients having chronic pain syndromes (e.g. Morbus Sudeck or neuralgias), even slight contacts or thermal stimuli can be perceived as unpleasant or even painful. If, in such cases, treatment has to take place via the contralateral extremity or face halves or body halves, the stimulus effect as a result of the application is not highly pronounced in the healthy body half. It is very advantageous in sensory CR stimulation overall if stimulation can take place using very small stimulus levels since sensory stimuli (e.g. tones, brightness fluctuations of transmission eyeglasses, etc.) can disturb the physiological stimulus processing.

(ii) To be able to have a stimulation that is as focal as possible in the electrical or magnetic stimulation of peripheral nerves and to be able to avoid side effects that are caused by the co-stimulation of adjacent structures (e.g. muscular contractions, sensations of pain, etc., it is important to use stimulus levels that are as small as possible.

(iii) Both the electrical and the magnetic stimulation of the brain or of the spinal cord are not very focal. The direct electrical stimulation of the brain in particular results in a co-stimulation of widely extensive brain areas that should actually be avoided or reduced at all costs with a chronic stimulation even in the most favorable cases of stimulation via a plurality of small electrodes and on the use of complex and/or expensive head models in addition to a focal strong stimulation. In this same manner, the ultrasound stimulation should be restricted to the actual target regions in the brain.

It is therefore necessary in all these cases to be able to treat with stimulus levels that are as small as possible to reduce the unwanted co-stimulation of non-target areas. However, this frequently has the result that the treatment is not sufficiently effective.

### SUMMARY

It is therefore the underlying object of the invention to provide an apparatus and a method that allow good, and in particular long-lasting, therapeutic effects by stimulation with highly minimal stimulation levels.

The object underlying the invention is satisfied by the features of the independent claims. Advantageous further developments and aspects of the invention are set forth in the dependent claims.

### BRIEF DESCRIPTION OF DRAWINGS

The invention will be described in more detail in the following in an exemplary manner with reference to the drawings. There are shown in these:

FIG. 1 illustrates a schematic representation of an apparatus for suppressing a pathologically synchronous and oscillatory neuronal activity and in particular for desynchronizing neurons having a pathologically synchronous and oscillatory activity in accordance with a first embodiment;

FIG. 2 illustrates a schematic representation of an apparatus for suppressing a pathologically synchronous and oscillatory neuronal activity and in particular for desynchronizing neurons having a pathologically synchronous and oscillatory activity in accordance with a second embodiment;

FIG. 3 illustrates a flowchart for illustrating a regulation of the lengths of stimulation phases and stimulation breaks in accordance with a first variant;

FIG. 4 illustrates a flowchart for illustrating a regulation of the lengths of stimulation phases and stimulation breaks in accordance with a second variant;

FIG. 5 illustrates a schematic illustration of an apparatus for the acoustic stimulation of neurons having a pathologically synchronous and oscillatory neuronal activity;

FIG. 6 illustrates a schematic illustration of an apparatus for the visual stimulation of neurons having a pathologically synchronous and oscillatory neuronal activity;

FIG. 7 illustrates a schematic representation of an apparatus for the tactile, vibratory, thermal, electrical transcutaneous and/or magnetic stimulation and/or ultrasound stimulation of neurons having a pathologically synchronous and oscillatory neuronal activity;

FIG. 8 illustrates a schematic representation of a CR stimulus sequence for stimulating a neural ensemble;

FIG. 9 illustrates graphs for illustrating an effective and an ineffective CR stimulation;

FIGS. 10 to 14 illustrate graphs for illustrating CR stimulations having different stimulation phase lengths and stimulation break lengths; and

FIG. 15 illustrates a diagram for representing the effectiveness of CR stimulations having different stimulation phase lengths and stimulation break lengths.

### DETAILED DESCRIPTION

An apparatus 1 for stimulating neurons having a pathologically synchronous and oscillatory neuronal activity is shown schematically in FIG. 1. The apparatus 1 comprises a control and analysis unit 10 and a stimulation unit 11. During the operation of the apparatus 1, the control and analysis unit 10 carries out a control of the stimulation unit 11. For this purpose, the control and analysis unit 10 generates control signals 21 which are received by the stimulation unit 11. The stimulation unit 11 generates stimuli 22 which are generated using the control signals 21 and which are administered to a patient. The stimuli 22 can be sensory stimuli, e.g. acoustic, visual, tactile, vibratory, thermal, olfactory, gustatory, magnetic and/or electrical transcranial, magnetic and/or electrical transcutaneous stimuli and/or ultrasound stimuli. The stimuli 22 can in particular be consciously perceivable by the patient. The stimuli 22 are adapted to suppress the pathologically synchronous and oscillatory neuronal activity on administration to the patient and in particular to desynchronize the neurons having the pathologically synchronous and oscillatory activity. The thermal stimuli 22 can in particular be produced by laser light.

The stimulus unit 11 and in particular also the control and analysis unit 10 are non-invasive units, i.e. they are located outside the body of the patient during the operation of the apparatus 1 and are not surgically implanted in the body of the patient.

During the operation of the apparatus 1, the efficiency of the stimulation can be improved at low stimulation levels, if an insufficient stimulation effect is determined, by the introduction of stimulation breaks, for example by the physician or by the user. No application of stimuli that could suppress the pathologically synchronous and oscillatory neuronal activity takes place during the stimulation breaks. It is, however, conceivable that different stimuli that are not adapted to suppress pathologically synchronous and oscillatory neuronal activity are applied during the stimulation breaks, in particular using the stimulation unit 11. In accordance with a further embodiment, stimulation of any kind with the aid of the stimulation unit 11 is dispensed with during the stimulation breaks. The above-described stimulation breaks can furthermore be added, e.g. in the case of side effects, to allow an efficient stimulation at low stimulation levels. The length of the stimulation breaks can be kept constant, can be set by the physician or user or can be regulated as described further below.

The invention utilizes a counter-intuitive relationship. The smaller the success achieved by the weak stimulation, the longer the breaks inserted into the stimulation procedure. It can in particular be brought about by the introduction of such breaks that an otherwise ineffective stimulation is effective.

The counter-intuitive mechanism underlying the invention can be made plausible by the following considerations. As a result of synaptic plasticity, assemblies of neurons are very plastic, i.e. they can be present in a plurality of different stable states. E.g. in states with a low mean synaptic connection strength and asynchronous neuronal activity, i.e. the neurons fire in an uncorrelated manner, or in states with a highly pronounced mean synaptic connection strength and synchronous neuronal activity, i.e. the neurons fire in a correlated manner, e.g. in time, that is coincident. There are typically a plurality of stable states having an intermediate mean synaptic connection strength and an intermediate pronounced state of the neuronal synchronization between these two extremes. Multistability in the mathematical sense is therefore present. The invention utilizes the surprising fact that the system, i.e. the stimulated neural ensemble, can be pushed from one attractor (stable state) to the next even with a low stimulation if there is a sufficiently long break between the stimulation phases during which the system is spontaneously pulled into the new attractor (i.e. without stimulation), which would not be possible under stimulation. The system moves so-to-say stepwise from highly synchronous attractors to increasingly weaker synchronous attractors due to the portioned stimulation.

The introduction of sufficiently long stimulation breaks allows an efficient stimulation at low stimulation levels. The stimulation level can then be smaller by a factor of 2 to 3 than the minimum stimulation level that results in a long-lasting desynchronization with permanent stimulation, i.e. with a stimulation without the stimulation breaks described herein. The stimulus level of the stimulation in accordance with the invention with stimulation breaks can in particular be in a range from ⅓ of the minimum stimulus level up to ⅔ of the minimum stimulus level that results in a long-lasting desynchronization on a permanent stimulation without the stimulation breaks in accordance with the invention.

The length of a stimulation break between two consecutive stimulation sections can amount to at least 3 minutes, but can also be substantially longer and can, for example, amount to at least 5 minutes or at least 10 minutes or at least 20 minutes or at least 30 minutes or at least 1 hour or at least 2 hours or at least 3 hours. To achieve first effects, the stimulation break length has to correspond to at least 200 periods of the oscillation to be desynchronized. A pronounced desynchronization can only be achieved from approximately 1,000 up to even 22,000 periods. In the case of a delta oscillation at a frequency in the range from 1 to 4 Hz, the period length amounts e.g. to 500 ms at 2 Hz. I.e. good effects result with breaks in the minute range or even in the hour range (1,000 to 22,000 periods then correspond to approximately 8.3 min or 3 hours). The period of pathological oscillation can for example be measured at the patient; but textbook values or experience values can also be used.

The length of the stimulation phases in which a stimulation takes place can furthermore preferably be set in addition to the length of the stimulation breaks to improve the efficiency of the stimulation at low stimulation levels. The length of the stimulation breaks can be kept constant in the same manner as the length of the stimulation phases, can be set by the physician or user or can be regulated as described further below.

The stimulation breaks can preferably be extended with too small a stimulation effect and the stimulation phases can likewise be extended.

The stimulation breaks and stimulation phases can, for example, each be of equal length and can thus increase equally. The stimulation phases can furthermore also be shorter at the start than the stimulation breaks and can increase disproportionately with too small a stimulation effect. Furthermore, any suitable other relation between the duration of the stimulation breaks and the duration of the stimulation phases can be set.

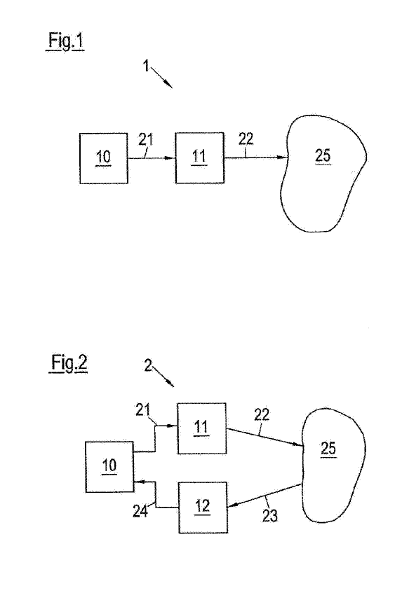

An apparatus 2 for stimulating neurons having a pathologically synchronous and oscillatory neuronal activity is shown schematically in FIG. 2. The apparatus 2 represents a further development of the apparatus 1 shown in FIG. 1. The apparatus 2, just like the apparatus 1, has a control and analysis unit 10 and a non-invasive stimulation unit 11. During the operation of the apparatus 1, the control and analysis unit 10 carries out a control of the stimulation unit 11. For this purpose, the control and analysis unit 10 generates control signals 21 which are received by the stimulation unit 11.

As described above, the stimulation unit 11 generates stimuli 22 with reference to the control signals 21 that are administered to a patient. The stimuli 22 can be sensory stimuli, e.g. acoustic, visual, tactile, vibratory, thermal, olfactory, gustatory, thermal and/or electrical transcranial, magnetic and/or electrical transcutaneous stimuli and/or ultrasound stimuli.

The apparatus 2 furthermore comprises a measuring unit 12. The stimulation effect achieved by the stimuli 22 is measured with the aid of the measuring unit 12. The measuring unit 12 records one or more measured signals 23 measured at the patient, converts them as required into electrical signals 24 and supplies them to the control and analysis unit 10. The neuronal activity in the stimulated target zone or in a zone associated with the target zone can in particular be measured by means of the measuring unit 12, with the neuronal activity of this zone correlating sufficiently closely with the neuronal activity of the target zone. A non-neuronal activity, e.g. a muscular activity, or the activation of the autonomous nervous system can also be measured by means of the measuring unit 12 provided that they are sufficiently closely correlated with the neuronal activity of the target region.

The measuring unit 12 includes one or more sensors that in particular make it possible to demonstrate a decrease or increase in the amplitude of the pathological oscillatory activity.

Non-invasive sensors can be used as the sensors, e.g. electroencephalograph (EEG) electrodes, magnetic encephalograph (MEG) sensors and sensors for measuring local field potentials (LFPs). The neuronal activity can also be determined indirectly by measurement of the associated muscular activity by means of electromyography (EMG) or indirectly by measuring the activation of the autonomous nervous system by means of measuring the skin resistance.

Alternatively, the sensors can be implanted in the body of the patient. Epicortical electrodes, deep brain electrodes for the measurement of e.g. local field potentials, subdural or epidural brain electrodes, subcutaneous EEG electrodes and subdural or epidural spinal cord electrodes can, for example, serve as invasive sensors.

The control and analysis unit 10 processes the signals 24, e.g. the signals 24 can be amplified and/or filtered, and analyzes the processed signals 24. The control and analysis unit 10 in particular controls the stimulation unit 11 with reference to the results of this analysis. The control and analysis unit 10 can include e.g. a processor (e.g. a microcontroller) for carrying out its work.

The control and analysis unit 10 checks the stimulation success with reference to the measured signals recorded in response to the application of the stimuli and sets the stimulation parameters, in particular the lengths of the stimulation breaks described above in connection with FIG. 1, in dependence on the stimulation success. In the case of side effects and/or generally with an insufficient stimulation effect, the efficiency of the stimulation can be improved in operation at low stimulus levels by the adaptation of the stimulation breaks. The duration of the stimulation breaks and the duration of the stimulation phases can be regulated with too small a stimulation effect such that a stimulation effect is again adopted.

The stimulation success can in particular be checked by means of a threshold value comparison. Depending on which signals are used for determining the stimulation success, different threshold value comparisons result. If e.g. the pathologically neuronal synchronization is measured via the sensors of the measuring unit 12, e.g. EEG electrodes, experience has shown that the lowering of the synchronization by e.g. at least 20% in comparison with the situation without stimulation is sufficient to determine a sufficient stimulation success. In accordance with an embodiment, an insufficient stimulation success can be determined if the pathologically neuronal synchronization by the application of the stimuli 22 is not reduced by at least a predefined value. If symptoms of the patient are used for determining the stimulation success, which reduction is to be considered as a clinically relevant improvement depends on the kind of clinical parameters used. Such reduction values (e.g. in the sense of the so-called minimal clinically perceptible improvement) are familiar to the skilled person.

Stimulation phases in which the brain and/or spinal cord 25 of the patent is/are stimulated by the stimuli 22 produced by the stimulation unit 11 and stimulation breaks in which no stimuli 22 are applied can be observed in alternating order.

Standard processes of bivariable control can e.g. be used for the regulation of the duration L_{Stim} of the stimulation phases and of the duration L_{Break} of the stimulation breaks. Medical a priori knowledge can, however, also be used, with the lengths L_{Stim} and L_{Break} being increased from a start value in the (L_{Stim}, L_{Break}) plane with a constant or successively increasing or deterministically and/or chaotically varied increment along a straight line or a bent curve. For example, the ratio L_{Stim}/L_{Break} can increase from 1/n to n within the framework of this regulation procedure, where n is, for example, a number in the range from 2 to 10, e.g. 3 or 4 or 5.

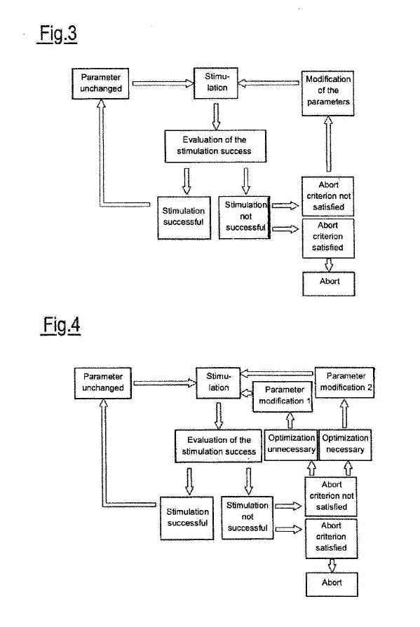

FIG. 3 shows a flowchart for an exemplary regulation of the lengths L_{Stim} and L_{Break} of the stimulation phases and stimulation breaks in accordance with a first variant. The lengths L_{Stim} and L_{Break} can, for example, be of equal size during the total process, i.e. L_{Stim}=L_{Break}=A applies. The ratio L_{Stim}/L_{Break} can, however, also be used as the variable for the regulation process. In the latter case, the length L_{Stim} can, for example, differ by up to ±5% or up to ±10%, or up to ±25% from the length L_{Break}, i.e. L_{Stim}=(1+ε)L_{Break}=A applies, wherein ε in the above-named cases amounts to up to ±0.05 or ±0.1 or ±0.25.

The parameter A is kept constant for so long from a preset starting value until the control and analysis unit 10 classifies the stimulation as unsuccessful. The parameter A is then in particular incrementally increased until the control and analysis unit 10 determines with reference to the measured signals 24 recorded by the measuring unit 12 that the stimulation is again sufficiently successful.

In accordance with an embodiment, the parameter A is increased, in particular incrementally, for so long with a non-sufficient stimulation success until a sufficient stimulation success is determined or an abort criterion is satisfied. The abort criterion should determine when no sufficient stimulation success is to be expected despite a sufficiently large and sufficiently justifiable effort.

The abort criterion can e.g. be satisfied when at least one of a plurality of criteria is satisfied. An abort criterion K1 can e.g. be satisfied if a predefined treatment duration, e.g. of 12 weeks, is exceeded and is otherwise not satisfied. The choice of the predefined treatment duration depends on the respective disease pattern or disease stage and reflects clinical experience.

FIG. 4 shows a flowchart for a further exemplary regulation of the lengths L_{Stim} and L_{Break} of the stimulation phases and stimulation breaks in accordance with a second variant. The regulation shown in FIG. 4 is in many parts identical to the regulation of FIG. 3, but is more complex in the following respect. It has been found empirically that the regulation shown in FIG. 3 works robustly. A considerable amount of time can, however, typically be saved when—as described above—the ratio L_{Stim}/L_{Break} increases within the framework of the regulation procedure, adapted to the stimulation success, from 1/n to n, where n is, for example, a number in the range from 2 to 10, e.g. 3 or 4 or 5. If this adaptation rule does not work sufficiently, the criterion “optimization required” is satisfied. In this case, the apparatus in accordance with the invention changes to the more robust regulation method shown in FIG. 3. Specifically, the criterion “optimization required” is analog to an abort criterion, i.e. no sufficiently highly pronounced therapeutic success is reached within a specific time or after a specific number of regulation steps.

In accordance with an embodiment, an optimization criterion K2 is satisfied when biomarkers and/or self-evaluation scales, e.g. mental state scales or quality of life scales that are input e.g. via a mobile device (such as an iPhone) and are correspondingly evaluated, measured by means of invasive and/or non-invasive sensors do not improve sufficiently. The sensors of the measuring unit 12 can be used for the invasive sensors used here and/or non-invasive sensors. Different forms of electrodes, e.g. deep electrodes or epicortical electrodes, can in particular be used as invasive sensors. Chronically or intermittently used EEG electrodes or accelerometers can e.g. be used as non-invasive sensors for the detection of characteristic movement patterns such as tremor, akinesia or epileptic fits. A biomarker is e.g. the spectral density in a characteristic frequency region familiar to the skilled person (e.g. the beta band running from approximately 8 to 30 Hz for Parkinson's patients) of the local field potential derived via deep electrodes.

If the biomarker or biomarkers or the self-evaluation scales (i) does/do not fall within a predefined time, e.g. 4 weeks, by a specific percentage from the starting value, e.g. 20% or 50% over a multi-day average depending on the disease or the stage of the disease, and/or (ii) has/have not fallen from the starting value by this percentage after the mth adaptation step of the parameter A, e.g. m=3, the criterion K2 is satisfied in accordance with an embodiment. Otherwise the criterion K2 is not satisfied. In this respect, the adaptation steps of A are constant or successively increasing or are varied deterministically and/or chaotically. The repetition number m corresponds to clinical experience, i.e. the time scale on which the therapeutic success can be adopted with the respective disease.

Instead of the regulations shown in FIGS. 3 and 4, a stimulation can also be carried out in which the lengths L_{Stim} of the stimulation phases and the lengths L_{Break} of the stimulation breaks are constant, i.e. L_{Stim}=L_{Break}=A or L_{Stim}=(1+ε)L_{Break}=A with ε in the range of ±0.05, ±0.1 or ±0.25, and the stimulation being carried out for so long with a constant A until the stimulation is classified as unsuccessful by the control and analysis unit 10, i.e. until an abort criterion such as described in connection with the regulation in accordance with FIG. 3 is satisfied. The stimulation is then ended. The apparatus 2 can then emit a corresponding message (“stimulation aborted”) to the patient, e.g. on a display or by a flashing pilot lamp or the like. This message can also be sent by radio, e.g. as a text message, an email or the like, to the physician. As an alternative to this, the treatment can also be continued on the reaching of the abort criterion; the corresponding message to the patient is then e.g. “please consult physician” and the text message/email is sent to the treating physician to advise him of the insufficient therapy.

The non-invasive stimulation, e.g. non-invasive CR stimulation, can be an “open loop” stimulation or a “closed loop” stimulation. In the case of the “closed-loop” stimulation, non-implanted sensors of the measuring unit 12, e.g. chronically or intermittently used EEG or EMG electrodes or MEG sensors or accelerometers (for the detection of characteristic movement patterns such as tremor, akinesia, epileptic fits) or electrodes for measuring the skin resistance, and/or less preferred, implanted sensors, e.g. deep electrodes, epicortical electrodes, are used to control the stimulation. These sensors can also be used (i) to implement the regulation of the stimulation phases and stimulation breaks, i.e. their adaptation to the therapeutic effect or (ii) to determine optimization or abort criteria such as described above in connection with FIGS. 3 and 4. Different sensors or different signals or different signal components or different dynamic biomarkers (optionally determined from the same starting signals) for the “closed loop” stimulation can also be used for the regulation of the lengths L_{Stim} and L_{Break} of the stimulation phases and stimulation breaks. In the closed-loop stimulation, relevant stimulation parameters such as the CR stimulation frequency, the intensity of the individual stimuli, the duration of the individual stimuli, the subgroup of active actuators of a larger total group are e.g. controlled, but also the kind of stimulation, e.g. CR stimulation versus stimulation using noise signals of different types (Gaussian noise, colored noise, etc.) as well as a requirement-controlled stimulation in which stimulation only takes place on the presence of measured pathological markers.

The individual components of the apparatus 1 and 2, in particular the control and analysis unit 10, the stimulation unit 11 and/or the measuring unit 12, can be separate from one another in a construction aspect. The apparatus 1 and 2 can therefore also be understood as systems.

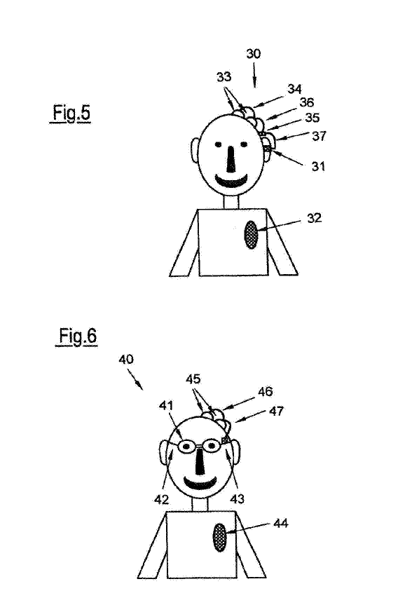

FIG. 5 schematically shows an apparatus 30 for non-invasive acoustic stimulation of neurons having a pathologically synchronous and oscillatory neuronal activity in accordance with an embodiment of the invention. Acoustic stimuli, in particular acoustic CR stimuli, are administered to the patient via earphones or headphones 31 with an earphone being a loudspeaker positioned in the ear canal. The control signals used for this purpose are generated by a control and analysis unit 32. Non-invasively fixed EEC electrodes 33 that are connected via a cable 34 serve for the “closed loop” stimulation and/or for the automatic above-described adaptation of the duration of stimulation breaks and stimulation phases. The corresponding calculation is carried out in a small component 35 that preferably contains a measurement amplifier and is connected to the EEG electrodes 33 or to the earphones or headphones 31 via cables 36, 37 and/or is carried out in the actual control and analysis unit 32 accommodating the battery or the rechargeable battery. The control and analysis unit 32 and the component 35 are connected to one another telemetrically in the embodiment shown in FIG. 5. In this case, the component 35 (or a component connected to it via cable) likewise contains a battery or a rechargeable battery. Alternatively, the control and analysis unit 32 and the component 35 can also be connected to one another via cable such that the component 35 is fed via the power supply of the control and analysis unit 32.

FIG. 6 schematically shows an apparatus 40 for the non-invasive visual stimulation of neurons having a pathologically synchronous and oscillatory neuronal activity in accordance with an embodiment of the invention. In this embodiment, the patient wears stimulation spectacles 41 that are e.g. fastened to the head of the patient via a clamp 42. A component 43 contains a calculation and telemetry unit. The latter serves for the connection to the actual control and analysis unit 44 accommodating the battery or the rechargeable battery. The component 43 and the control and analysis unit 44 are connected to one another telemetrically; in this case, the component 43 (or a component connected to it via cable) likewise contains a battery or a rechargeable battery. Alternatively, the component 43 and the control and analysis unit 44 can also be connected to one another via cable. Non-invasively fixed EEG electrodes serve for the “closed-loop” stimulation and/or the above described automatic adaptation of the duration of stimulation breaks and stimulation phases. The EEG electrodes 45 are connected to the component 43 via cables 46, 47.

The visual stimuli 41 generated by the stimulation spectacles 41 can have an underlying luminosity variation or brightness variation (or variation of the light intensity or luminosity); for example, they can be applied as pulses or as sequences of pulses with varied luminosity or brightness. The visual stimuli can be administered depending on the configuration as luminosity modulation of natural visual stimuli, e.g. by means of homogenous or segmented transmission spectacles in which the transmission can be regulated independently of the voltage, as a modulated visual stimulus occurring in addition to a natural visual stimulus, e.g. by means of partially transparent light spectacles or as an artificial visual brightness stimulus, e.g. by means of opaque light spectacles. The stimulation spectacles 41 are preferably divided into different segments whose luminosity or transmission or brightness can be controlled separately to be able to stimulate different points of the retina independently of one another.

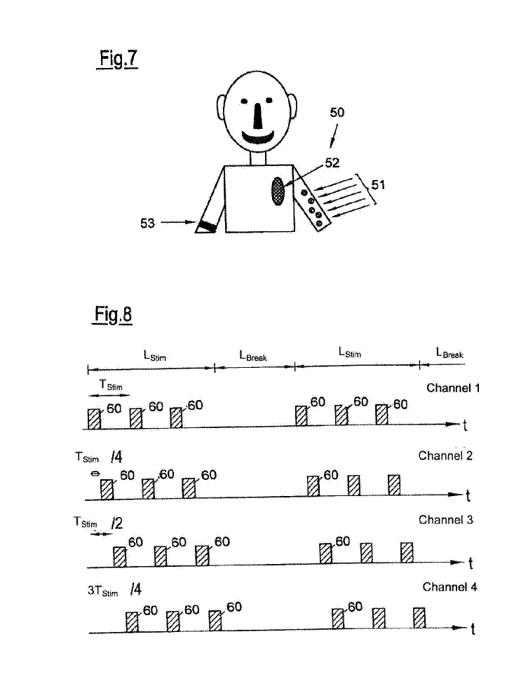

FIG. 7 schematically shows an apparatus 50 for the non-invasive tactile, vibratory, thermal, electrical transcutaneous and/or magnetic stimulation and/or for the ultrasound stimulation of neurons having a pathologically synchronous and oscillatory neuronal activity in accordance with an embodiment of the invention. The apparatus 50 comprises a stimulation unit 51, a control and analysis unit 52 controlling the stimulation unit 51, and an accelerometer 53 for recording measurement signals. The stimulation unit 51 and the accelerometer 53 can be connected to the control and analysis unit 52 telemetrically or via cable.

The stimulation unit 51 comprises a plurality of stimulation elements for generating tactile, vibratory, thermal, electrical transcutaneous and/or magnetic stimuli and/or ultrasound stimuli. The stimulation elements are designed such that they can be placed on the skin of the patient. Depending on the disease and/or on the effected parts of the body, the stimulation elements are secured to the skin of the patient in a suitable arrangement, for example to the arm, to the leg, to the hand and/or to the foot of the patient. The plurality of stimulation elements make it possible to stimulate different receptive regions of the skin via the individual stimulation elements with time and space coordination.

On the application of acoustic or visual stimuli, they are received via at least one ear or at least one eye of the patient. The tactile, vibratory, thermal, electrical transcutaneous and/or magnetic stimuli and/or ultrasound stimuli are received by receptors disposed in or under the skin and are forwarded to the nervous system. These receptors include, for example, Merkel cells, Ruffini corpuscles, Meissner's corpuscles and hair follicle receptors which in particular act as receptors for the tactile stimuli. The vibratory stimuli are predominantly directed to deep sensibility. The vibratory stimuli can be received by receptors disposed in the skin, in the muscles, in the subcutaneous tissue and/or in the sinews of the patient. Pacini's corpuscles, which communicate vibration perceptions and accelerations, can be named by way of example as receptors for the vibration stimuli. The thermal stimuli are received by the thermoreceptors of the skin. They are warm receptors (also called heat receptors, warm sensors or heat sensors) and cold sensors (also called cold receptors). The cold sensors are more superficial in the skin of people; the heat receptors somewhat lower. The electrical transcutaneous and magnetic stimuli and the ultrasound stimuli do not act specifically on only one group of receptors disposed in or under the skin. The target zone can therefore be stimulated via different channels using the electrical transcutaneous stimuli.

The directed stimulation of specific regions of the brain or spinal cord is made possible by the tonotopic or somatotopic association of body regions with these regions. For example, acoustic stimuli are converted into nerve impulses in the inner ear and are forwarded via the acoustic nerve to the auditory cortex. A specific portion of the auditory cortex is activated on the acoustic stimulation of the inner ear at a specific frequency due to the tonotopic arrangement of the auditory cortex.

In the visual stimulation, different points in the visual field are imaged on different points of the retina via the crystalline lens of the eye. The different points of the retina are in turn connected via the optic nerve to different neurons in the brain. Consequently, respective different neurons can be stimulated using the stimuli applied at different spatial sites.

Due to the somatotopic structuring of the neural pathways and of the associated zones of the brain, different neurons are furthermore stimulated by tactile, vibratory, thermal, electrical transcutaneous and/or magnetic stimuli and/or ultrasound stimuli which are applied at different sites on the skin. With these types of stimulation, the stimulation elements can be attached, for example, to the foot, lower leg and upper leg or to the hand, the lower arm and upper arm of the patient in order thereby to be able to stimulate specific neurons.

The stimulation units described above can accordingly separately stimulate different regions of the brain or spinal cord in that the applied stimuli are forwarded via neural conductors to different target zones which lie in the brain and/or spinal cord. The target zones can be stimulated with possibly different and/or time-offset stimuli during the stimulation.

The apparatus described herein, in particular the apparatus 1, 2, 30. 40 and 50 can in particular be used for treating neurological or psychiatric diseases, e.g. Parkinson's disease, essential tremor, tremor resulting from multiple sclerosis as well as other pathological tremors, dystonia, epilepsy, depression, locomotor disorders, cerebellar diseases, obsessive compulsive disorders, dementia, Alzheimer's, Tourette's syndrome, autism, functional disorders after stroke, spasticity, tinnitus, sleep disorders, schizophrenia, irritable bowel syndrome, addiction diseases, borderline personality disorder, attention deficit syndrome, attention deficit hyperactivity syndrome, pathological gambling, neuroses, bulimia, anorexia, eating disorders, burnout syndrome, fibromyalgia, migraine, chronic pain syndromes, cluster headache, general headache, neuralgia, ataxia, tic disorder or hypertension as well as further diseases which are characterized by pathologically increased neuronal synchronization.

The aforesaid diseases can be caused by a disorder of the bioelectric communication of neural assemblies which are connected in specific circuits. In this respect, a neural ensemble continuously generates pathological neuronal activity and possibly a pathological connectivity associated therewith (network structure). In this respect, a large number of neurons synchronously form action potentials, i.e. the participating neurons fire excessively synchronously. In addition, there is the fact that the pathological neural ensemble has an oscillatory neuronal activity, i.e. the neurons fire rhythmically. In the case of neurological or psychiatric diseases, the mean frequency of the pathological rhythmic activity of the affected neural assemblies lies approximately in the range from 1 to 50 Hz, but can also be outside this range In healthy people, the neurons fire qualitatively differently, however, e.g. in an uncorrelated manner.

In the above-mentioned CR stimulation, the stimuli administered to the patient are forwarded via the nervous system to a neural ensemble in the brain and/or spinal cord that has a pathologically synchronous and oscillatory neuronal activity. The stimuli are designed such that the pathologically synchronous activity of the neural ensemble is desynchronized. A lowering of the coincidence rate of the neurons effected by the stimulation can result in a lowering of the synaptic weights and thus in an unlearning of the tendency to produce pathologically synchronous activity.

The stimuli administered in the CR stimulation effect a reset of the phase of neuronal activity of the stimulated neurons in the neural ensemble. The phase of the stimulated neurons is set to or close to a specific phase value, e.g. 0°, independently of the current phase value by the reset (it is not possible in practice to set a specific phase value exactly; however, this is also not required for a successful CR stimulation. The phase of the neuronal activity of the pathological neural ensemble is thus monitored by means of a direct stimulation. Furthermore, the pathological neural ensemble is stimulated by means of a plurality of stimulation contacts of the stimulation unit at different points such that the phase of neuronal activity of the pathological neural ensemble can be reset at the different stimulation points at different points in time. As a result, the pathological neural ensemble whose neurons were previously active synchronously and at the same frequency and phase are split into a plurality of subpopulations. Within each of the subpopulations, the neurons are still synchronous after the resetting of the phase and also still fire at the same pathological frequency, but each of the subpopulations has the phase with respect to their neuronal activity which was enforced by the stimulus generated by the respective stimulation contact. This means that the neuronal activities of the individual subpopulations still have the same pathological frequency, but different phases, after the resetting of their phases into an approximately sinusoidal curve.

Due to the pathological interaction between the neurons, the state with at least two subpopulations generated by the stimulation is unstable and the total neural ensemble fast approaches a state of complete desynchronization in which the neurons fire without correlation. The desired state i.e. the complete desynchronization is thus not immediately present after the time-offset (or phase-shifted) application of the phase-resetting stimuli, but is usually adopted within a few periods or even in less than one period of the pathological frequency.

FIG. 8 shows an example of a CR stimulation with a total of four channels. A subpopulation of the pathological neural ensemble is stimulated over each of the four channels. In each of the four channels, pulse-shaped stimuli 60 are applied in a sequence periodically with the period T_{Stim}, where T_{Stim} is here also close to the middle period of the pathological oscillation of the neural ensemble or deviates from the textbook value by up to ±5%, ±10% or ±20% (typically f_{Stim}=1/T_{Stim} lies in the range from 1 to 30 Hz). The stimuli 60 effect a phase reset of the neuronal activity of the respective stimulated subpopulation. The time delay between the sequences of adjacent channels furthermore amounts to T_{Stim}/4, since four channels are present. For the general case of N channels, the time delay of adjacent channels would amount to T_{Stim}/N (it is also possible to deviate from this value by e.g. up to ±5%, ±10% or ±20%). Furthermore, the sequence of the stimulus administration over the N channels does not have to be identical in each stimulation cycle, but can e.g. also be varied in a randomized manner from stimulation cycle to stimulation cycle.

Depending on the modality of the stimuli 60, they are configured such that the respective desired subpopulation is stimulated. In the case of acoustic stimulation, the stimuli 60 in a respective channel, for example, have a specific frequency or a specific frequency range that is selected such that a specific subpopulation is stimulated due to the tonotropic organization of the auditory cortex. In the case of visual stimulation, the channels correspond to different points in the visual field that are imaged via the crystalline lens of the eye at different points of the retina that are in turn connected to different neurons in the brain via the optic nerve. In the case of tactile, vibratory, thermal, electric transcutaneous and/or magnetic stimuli 60 and/or ultrasound stimuli 60, the channels stand for different points of the skin at which the stimuli 60 are applied and which re connected to the desired subpopulations in the brain or spinal cord via the nervous system.

Furthermore, in FIG. 8, the lengths L_{Stim} of the stimulation phases and the lengths L_{Break} of the stimulation breaks are shown that can be set or regulated as described above. It must be noted that in FIG. 8, the lengths L_{Stim} and L_{Break} and the lengths of the phase-reset stimuli 60 are not reproduced true to scale.

It must be pointed out that breaks in which no stimuli are applied can also be observed in conventional stimulation methods. For example, in CR stimulation, stimulation can take place for n cycles and no stimulation can take place for the following m cycles and this stimulation pattern can be periodically continued, where n and m are small whole numbers. Such breaks can also be observed in accordance with the invention during the stimulation phases of the length L_{Stim}. The stimulation breaks in accordance with the invention of the length L_{Break}, however, differ from the breaks during the stimulation phases in that they are only observed when it was previously found that the stimulation success achieved by the stimulation is not sufficient and/or if side effects occur and/or if the stimulation unit is unfavorably positioned in the body of the patient.

Different stimulation forms instead of CR stimulation can also be used provided that long-lasting therapeutic effects can be achieved with these stimulation forms in the desynchronization of pathologically active neural ensembles.

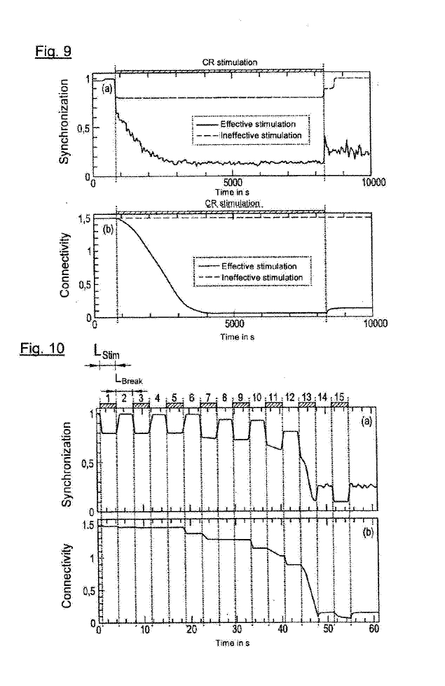

The effects achievable using the invention described herein are illustrated with reference to simulation results in FIGS. 9 to 15.

In FIGS. 9(a) and 9(b), the degree of synchronization and the synaptic connectivity of a neural ensemble having a pathologically synchronous and oscillatory neuronal activity are shown before, during and after a CR stimulation. The horizontal bars drawn at the top in both representations indicate the time period in which the CR stimulation is applied.

As FIGS. 9(a) and 9(b) show, an effective CR stimulation effects a fast desynchronization of the neural ensemble and a fast reduction in the connectivity. However, under certain circumstances, only a small stimulation success can arise, which can be seen from the fact that the degree of synchronization and the connectivity within the stimulated neural ensemble only reduce slightly despite the CR stimulation.

The efficiency of the CR stimulation can be improved with the above-described apparatus 1 and 2 by the insertion of stimulation breaks at low stimulus levels. Stimulation breaks can furthermore be added, e.g. in the case of side effects to allow an efficient stimulation at low stimulation levels.

Side effects depend on the respective disease and on the respectively selected target region. Dyskinesias can occur e.g. that present by a coactivation (instead of an alternating activation) of antagonistic muscles (e.g. flexors and extensors). Side effects can also present by a stimulation-dependent increase in synchronous activity in corresponding sensors.

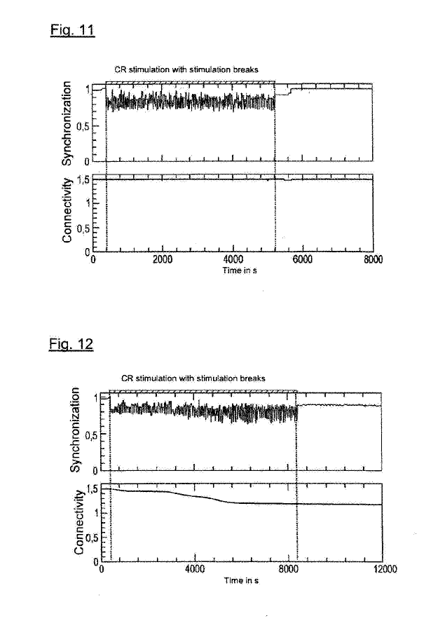

FIGS. 10(a) and 10(b) show the results of a stimulation that comprises alternating stimulation phases in which a CR stimulation is carried out and stimulation breaks in which no stimulation is carried out. The stimulation phases are drawn by horizontal bars in FIGS. 10(a) and 10(b). The lengths L_{Stim} and L_{Break} of the stimulation phases and stimulation breaks are of equal length and respectively amount to 3,600 s in the present example. Except for the stimulation breaks, the same stimulation parameters were used for the simulations shown in FIGS. 10(a) and 10(b) as for the simulation of the ineffective stimulation shown in FIGS. 9(a) and 9(b). The insertion of the stimulation breaks produces a clear reduction in the degree of synchronization and in the connectivity. The insertion of the stimulation breaks consequently has the effect that an otherwise ineffective stimulation is effective. Furthermore, long-lasting therapeutic effects can be achieved with this form of stimulation. The degree of synchronization and the connectivity also remain at a very low level after the complete switching off of the stimulation.

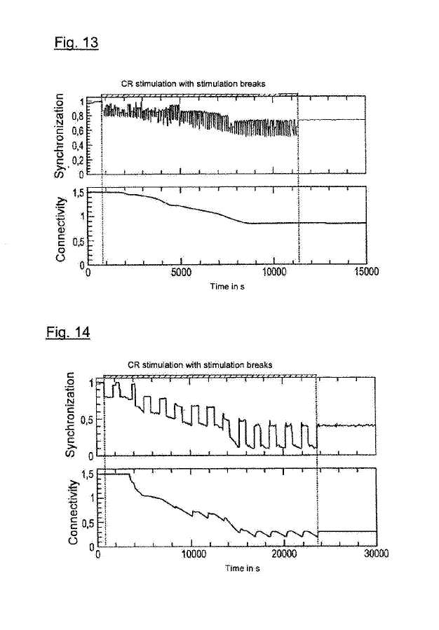

It is important for the success of the form of stimulation in accordance with the invention to determine suitable lengths L_{Stim} and L_{Break} for the stimulation phases and stimulation breaks. FIGS. 11 to 14 show the results of different simulations for which different values for L_{Stim} and L_{Break} were used with otherwise the same stimulation parameters. The values are shown in the following table.

| | L_{Stim} | L_{Break} |
|---|---|---|
| FIG. 11 | 24 s | 24 s |
| FIG. 12 | 24 s | 56 s |
| FIG. 13 | 24 s | 80 s |
| FIG. 14 | 960 s | 720 s |

In FIGS. 11 to 14, those time periods are marked by the horizontal bars in which the CR stimulation in accordance with the invention is carried out with mutually alternating stimulation phases and stimulation breaks.

For only very brief stimulation phases and stimulation breaks, only a very small effect is produced (cf. FIG. 11) that is furthermore of only brief duration after the ending of the stimulation process. Better results are achieved when, with an unchanging length L_{Stim} of the stimulation phases, the length L_{Break} of the stimulation breaks is increased (cf. FIGS. 12 and 13). The best results are achieved in the simulations shown here for relatively large values of several minutes for the lengths L_{Stim} and L_{Break} (cf. FIG. 14).

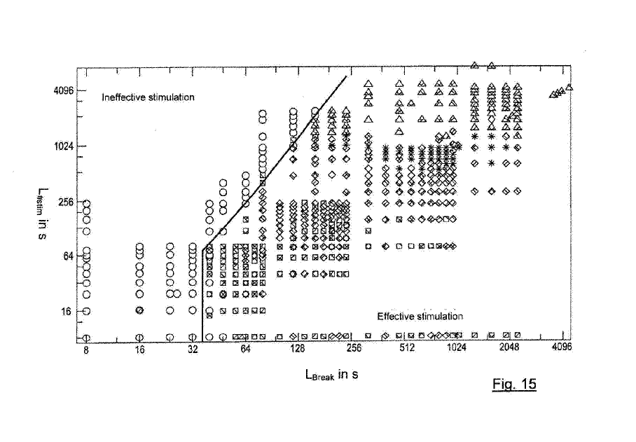

In FIG. 15, the results of different CR stimulations in accordance with the invention are shown in an (L_{Stim}/L_{Break}) plane. The circular symbols show ineffective stimulations; all the other symbols represent effective stimulations. Furthermore the (L_{Stim}/L_{Break}) plane in FIG. 14 is divided by a line into a region of ineffective stimulation and a region of effective stimulation. As can be seen from FIG. 14, stimulations having only short values for L_{Stim} and L_{Break} as well as stimulations in which L_{Stim} is too long in comparison with L_{Break} are ineffective. The best stimulation results were achieved for parameter values from the top right region of the (L_{Stim}/L_{Break}) plane.

1-15. (canceled)

16. An apparatus for suppressing a pathologically synchronous and oscillatory neuronal activity, the apparatus comprising
a non-invasive stimulation unit for stimulating neurons, using stimuli, in at least one of a brain and a spinal cord of a patient that demonstrate a pathologically synchronous and oscillatory neuronal activity, wherein the stimuli are adapted to suppress the pathologically synchronous and oscillatory neuronal activity when administered to the patient;
a measuring unit for recording measured signals that reproduce a neuronal activity of the stimulated neurons; and
a control and analysis unit for controlling the stimulation unit and for analyzing the measured signals, wherein the control and analysis unit is configured to:
control the stimulation unit to apply the stimuli,
determine success of the stimulation with reference to the measured signals recorded in response to the application of the stimuli, and
if the success of the stimulation is not sufficient, insert at least one stimulation break between application of the stimuli or extend at least one stimulation break, wherein no stimuli are applied during the at least one stimulation break that can suppress the pathologically synchronous and oscillatory neuronal activity.

17. The apparatus in accordance with claim 16, wherein the stimuli are selected from at least one of acoustic, visual, tactile, vibratory, thermal, olfactory, gustatory, magnetic transcranial, electrical transcranial, magnetic transcutaneous, electrical transcutaneous stimuli, and ultrasound stimuli.

18. The apparatus in accordance with claim 16, wherein the control and analysis unit is furthermore configured to extend the duration of the at least one stimulation break until the control and analysis unit determines that the success of the stimulation is sufficient.

19. The apparatus in accordance with claim 18, wherein the control and analysis unit is further configured to incrementally extend the duration of the at least one stimulation break.

20. The apparatus in accordance with claim 18, wherein the control and analysis unit is furthermore configured to extend the duration of stimulation phases, in addition to the duration of the at least one stimulation break, until the control and analysis unit determines that the success of the stimulation sufficient.

21. The apparatus in accordance with claim 20, wherein the control and analysis unit is further configured to incrementally extend the duration of the stimulation phases.

22. The apparatus in accordance with claim 16,
wherein the stimulation phases each have a duration L_{Stim} and the at least one stimulation break observed between mutually following stimulation phases each have a duration L_{Break} and A=L_{Stim}=L_{Break} or A=L_{Stim}/L_{Break} applies, and
wherein the control and analysis unit is furthermore configured to either increase A or increase A incrementally, if the success of the stimulation is not sufficient.

23. The apparatus in accordance with claim 22, wherein the control and analysis unit is furthermore configured to either increase A for A=L_{Stim}/L_{Break} from 1/n to n or increase A for A=L_{Stim}/L_{Break} from 1/n to n incrementally, if the success of the stimulation is not sufficient.

24. The apparatus in accordance with claim 23, wherein n is a number in the range from 2 to 10.

25. The apparatus in accordance with claim 22, wherein the control and analysis unit is furthermore configured to keep A constant for so long until the control and analysis unit determines that the success of the stimulation is not sufficient.

26. The apparatus in accordance with claim 22, wherein the control and analysis unit is furthermore configured to increase A with an insufficient stimulation success for so long until the control and analysis unit determines that the success of the stimulation is sufficient or until an abort criterion is satisfied.

27. The apparatus in accordance with claim 16, wherein the stimulation unit is configured to perform a coordinated reset stimulation during the stimulation phases, with the phases of neuronal activity of a plurality of subpopulations of a stimulated neural ensemble having a pathologically synchronous and oscillatory neuronal activity being reset at different points in time.

28. The apparatus in accordance with claim 16, wherein the duration of the at least one stimulation break is at least 3 minutes.

29. The apparatus in accordance with claim 16, wherein the duration of the at least one stimulation break corresponds to a duration of at least 200 or at least 1,000 periods of the pathologically synchronous and oscillatory neuronal activity.

30. A method for suppressing a pathologically synchronous and oscillatory neuronal activity, the method comprising:
stimulating, by a non-invasive stimulation unit, at least one of a brain and a spinal cord of a patient that demonstrate a pathologically synchronous and oscillatory neuronal activity, using at least one of electrical and optical stimuli, wherein the stimuli are adapted to suppress the pathologically synchronous and oscillatory neuronal activity on an administration to the patient; and
if at least one of stimulation success achieved by the stimulation is not sufficient and side effects occur to the patient, inserting at least one stimulation breaks into the application of the stimuli or extended the at least one stimulation break, with no stimuli being applied during the stimulation breaks that could suppress the pathologically synchronous and oscillatory neuronal activity.

31. The method in accordance with claim 30, further comprising:
recording measured signals that reproduce a neuronal activity of the stimulated neurons; and
determining, the stimulation success with reference to the measured signals recorded in response to the application of the stimuli.

32. The method in accordance with claim 30, further comprising extending incrementally the duration of the at least one stimulation break incrementally until the stimulation success is sufficient.

33. The method in accordance with claim 32, further comprising extending incrementally the duration of the at least one stimulation break.

34. The method in accordance with claim 32, further comprising extending a duration of stimulation phases, in addition to the duration of the at least one stimulation break, until the stimulation success is sufficient.

35. The method in accordance with claim 34, further comprising incrementally extending the duration of the stimulation phases.
